(19) United States
(12) Patent Application Publication
(10) Pub. No.: US 2025/0146059 A1
(43) Pub. Date: May 8, 2025

(54) Method and Reagent Kit for Targeted Genomic Enrichment

(71) Applicant: Advancedseq LLC, Livermore, CA (US)

(72) Inventor: Zhaohui ZHOU, San Ramon, CA (US)

(21) Appl. No.: 18/936,001

(22) Filed: Nov. 4, 2024

Related U.S. Application Data
(60) Provisional application No. 63/596,263, filed on Nov. 4, 2023.

(51) Int. Cl. C12Q 1/6848; C12N 9/22; C12N 15/11; C12Q 1/25; C12Q 1/44; C12Q 1/48; C12Q 1/6806; C12Q 1/6853

(52) U.S. Cl. CPC C12Q 1/6848; C12N 9/22; C12N 15/111; C12Q 1/25; C12Q 1/44; C12Q 1/485; C12Q 1/6806; C12Q 1/6853; C12N 2310/20; G01N 2333/9015; G01N 2333/9126; G01N 2333/922

(57) ABSTRACT

A method for cleaving a target DNA to isolate a target DNA fragment sequence of interest, directed by a targeting oligonucleotide followed by enriching the target DNA fragment sequence is disclosed. The targeting oligonucleotide binds to the target DNA during DNA cleavage. After cleavage, the target DNA fragment sequence of interest is modified using either a ligation or a polymerase extension method. The resulting target DNA fragment sequence of interest is enriched by exonuclease treatment.

This application claims the benefit of priority to U.S. Provisional application No. 63/596,263, filed Nov. 4, 2023, entitled “METHOD AND REAGENT KIT FOR TARGETED GENOMIC ENRICHMENT”, and which is hereby incorporated by reference in its entirety. All references cited and discussed in this specification are incorporated herein by reference in their entireties and to the same extent as if each reference were individually incorporated by reference.

### FIELD OF THE DISCLOSURE

This invention relates to methods and reagent kits for isolating and amplifying dsDNA fragments with sequence specificity from a larger dsDNA piece or genomic dsDNA. One of the applications of the invention is for targeted genomic enrichment, for example, to isolate a DNA region of interest from the whole genome for DNA sequencing.

### BACKGROUND

The advancement in next-generation sequencing technologies has improved our ability to sequence large genomes at a lower cost and faster speed than ever before. However, it is still not feasible to apply whole genome sequencing routinely in clinical settings. The primary reason is that the cost and time of sequencing the entire genome with an accuracy level sufficient to call the variant of interest is still prohibitively high. Contrary to common conception that a person only needs to have his/her gene sequenced once in a lifetime, sequencing may be required multiple times each for a specific purpose. For examples, in cancer diagnostics, heterogeneous cell populations such as tumor cells and normal cells would be sequenced at the same time. In analyzing disease progression, cells from the same source may need to be sequenced at different times. Sequencing may also be applied in prenatal diagnostics to specific cell populations.

In many applications, the goal is only to get an accurate picture of a certain region or regions of the genome of these particular cell populations. Without isolating the specific genomic region, whole genome sequencing is not only wasteful, but also causes delay and inaccuracy. Therefore, a genomic enrichment method that allows isolation of a specific region or regions of interest will lower the cost of sequencing, improve accuracy, and cut time to result significantly.

A number of methods have been used for genome enrichment. One method is PCR based, in which multiple PCR primers are designed and tested. However, PCR amplification and normalization process is labor intensive, and as a result, this method cannot be applied universally. In addition, PCR can only be used for DNA fragments of certain limited size ranges, and complexity of the genome makes it hard to achieve high multiplex PCR with consistent result. A second method is based on sequence specific ligation followed by universal PCR. Again, ligation probe design, process optimization, and size limitation make it less than ideal. A third method is microarray hybridization based. The genomic DNA is sheered into small pieces, and a subset of genomic DNA sequences is captured based on complementary sequence identity. The captured DNA fragments are then taken through the typical library construction protocol.

A common characteristic of the existing targeted genomic enrichment methods is that the DNA region of interest, if more than a few hundred bases long, is captured in small fragments no more than a few hundred bases long. In PCR based methods, the length of each fragment is limited by the ability to reliably and consistently PCR amplify the fragment and is generally a few hundred bases long. In hybridization methods, the genomic DNA is randomly sheared into pieces of about a few hundred bases long, and then each piece is captured through hybridization. There are many inherent problems with capturing a long DNA region of interest in small fragments: (1) not all fragments are captured with the same efficiency, and some fragments may be missed altogether, and (2) many probes will have to be designed and made to cover the entire length of the region of interest, resulting in higher cost. Additionally, PCR may introduce errors to the amplified fragments. For hybridization, the specificity is low, and the processing time is long.

### SUMMARY

The key to overcoming the shortcomings of the existing targeted genomic enrichment methods is to be able to specifically cleave and isolate a long DNA region of interest in large fragments, preferably in one whole piece, rather than isolating many short fragments like in current methods. This requires the ability to (1) cleave a target DNA with sequence specificity at predetermined sites, and (2) isolate the cleaved DNA region of interest.

Described herein are methods and reagent kit for cleaving and purifying a DNA fragment of interest 5×10²−1×10⁸-base pairs long, enabling targeted genomic enrichment and selective genomic sequencing with higher specificity, simpler work flow, and lower cost. Central to the invention is a sequence specific DNA nuclease that is capable of cleaving the target DNA with sequence specificity, the protection of targeted DNA segments after the nuclease cleavage, the removal of non-target DNAs using exonuclease, and the isolation of the cleaved DNA fragment. Sequence specific means that the engineered nuclease is capable of cutting DNA with sequence specificity of eight base pairs or better. A specific sequence must be present for the engineered nuclease to cut. The cutting point may or may not be precisely at any particular base, but will be at close to where it is directed by the targeting sequence. Non-specific background cutting may also be present.

In an embodiment of the invention, a method utilizes a sequence specific DNA nuclease. The nuclease includes one or more targeting oligonucleotides. The nuclease is capable of cutting a target double-stranded DNA with sequence specificity greater than eight base pairs long. The cleaved DNA have cohesive end. The cleaved DNA segment of interest has cohesive ends flanking the target DNA. In another embodiment, a method for cutting out a DNA fragment of interest from a target DNA includes: cleave target DNAs with a sequence specific DNA nuclease described above; modified nucleotides are added to the cohesive ends by polymerases, or linkers with modified bases are added to the ends by ligases; removing non-modified DNAs with exonucleases; and tagging enriched target DNAs for further purification and manipulations.

In yet another embodiment, a reagent kit includes a sequence specific DNA nuclease, wherein the DNA nuclease is capable of cutting a target double-stranded DNA with sequence specificity greater than eight base pairs long; a polymerase wherein is capable of adding modified nucleotides to the cohesive ends; or a ligase wherein is capable of adding linkers with modified bases to the target DNAs; a exonuclease wherein is capable of removing non-target DNAs from the reaction mix; and an enzyme wherein is capable of adding tags to the enriched target DNAs.

### DESCRIPTION

### Brief Description of the Drawings

FIG. 1 is a diagram of the disclosed enrichment method for large genomic dsDNA segment(s) for either procaryotes or eucaryotes.

FIG. 2A depicts a schematic diagram of sequence specific nuclease gRNA/Cas12a proteins binding to a hypothetical target DNA.

FIG. 2B depicts the target DNA 112 cleaved at both ends by incubating with the CRISPR/Cas12a (102 and 102′), a programmable sequence specific DNA endonuclease or variants thereof.

FIG. 2C illustrates cohesive ends of double-stranded DNAs 114 with unpaired (single-stranded) DNA nucleotides on either the 5′ or 3′-strand, known as overhangs.

FIG. 2D illustrates modified 105 and native 105′ nucleotides filing in the overhanging ends of cleaved DNA segment of interest(s) by polymerase(s) 106 to provide the complementary strand.

FIG. 3 illustrates the experiment results performed as depicted in FIG. 2 demonstrating protection of two Plasmid dsDNA fragments generated after gRNA/Cas12a cleavages.

FIG. 4 illustrates the structural diagram of the human ABL proto-oncogene 1 (ABL1).

FIG. 5A represents the results following enrichment of an ABL1 DNA segment isolated from a human genomic dsDNA sample with a GAPDH used as a control in a TaqMan qPCR reaction before enrichment.

FIG. 5B illustrates that after exonuclease III treatment only the ABL1 gene was un-affected in a multiplexing assay and was enriched at least 100-fold relative to GAPDH as demonstrated in Example II.

### DETAILED DESCRIPTION OF THE EMBODIMENTS

### General Definitions

The term “Cas12a-associated guide RNA” refers to the RNA oligonucleotides that binds Cas12a protein and recognizes the target DNA region of interest and directs the Cas12a nuclease there for editing.

The term “Cas12a protein” refers to the Cas12a (CRISPR associated protein 12a, previously known as Cpf1) and it is a subtype of Cas12 proteins and forms part of the CRISPR system in some bacteria and archaea.

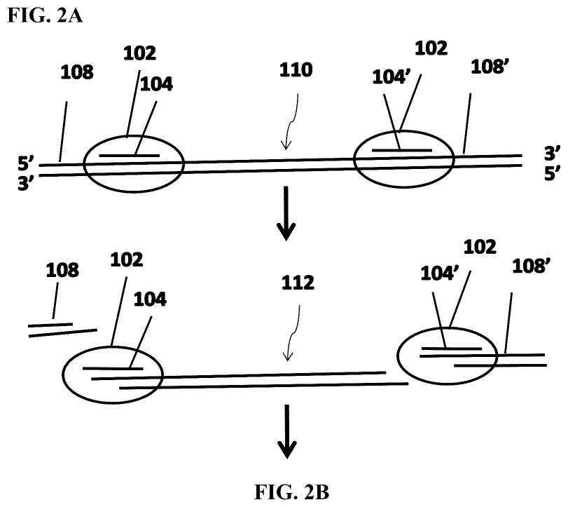

The terms “cohesive” and “cohesive ends” refers to double-stranded DNAs (dsDNAs) that have unpaired (single-stranded) DNA nucleotides on either the 5′- or 3′-strand, and are known as overhangs. This is illustrated in FIG. 2C with 105 and 105′, modified and natural nucleotides, respectively, filing in the overhanging ends to provide the complementary strand.

The terms “cut,” “cutting,” cleave,” and “cleaved” refers to making breaks in dsDNA strands through the sugar-phosphate backbone of DNA strands.

The term “enrich” or “enriched” refers to an increase in the concentration of a DNA fragment of interest compared to the non-interested DNAs within a sample of DNA.

The term “exonuclease” refers to an enzyme capable to digesting single-and/or double-stranded DNA Including, but not limited to exonuclease III, T7 exonuclease, exonuclease V, exonuclease VIII, Lambda exonuclease, T5 exonuclease, nuclease Bal-31, their variants and truncated forms and combinations thereof.

The term “genomic DNA” as used herein refers to double-stranded DNA (dsDNA) from a cell, tissue or culture sample of procaryotic or eukaryotic origin.

The term “guide RNA(s)” (gRNA) refers to a piece of RNA that functions as a guide for RNA- or DNA-targeting enzymes, e.g., including but not limited to CRISPR endonucleases, with which it forms complexes. The complexes are able to cleave DNA as specific sites on, within, at or flanking a target DNA sequence within a genomic DNA sample.

The terms “ligase” and “ligases” refers to an enzyme(s) capable of attaching an oligonucleotide and/or nucleotide sequence to either the 5′ or 3′ end of a DNA and/or RNA molecule or sequence. The ligase can include, but is not limited to, T4 DNA ligase, T4 DNA ligase 2, T4 RNA ligase1, T4 RNA ligase 2, SplintR ligase, RtcB ligase, T3 DNA ligase, Taq DNA ligase, 9° N DNA ligase, E. coli DNA ligase, as well as their variants and truncated forms and combinations thereof.

The term “ligate” refers to the covalent linking of two ends of DNA or RNA molecules.

The term “linkers” refers to the double-stranded DNA or RNA molecules that can be covalently linked to the ends of double-stranded DNA or RNA molecules.

The term “modified” refers to oligonucleotides, nucleotides and the like that are chemically modified in their triphosphate moiety, sugar moiety, or in their bases. These nucleotides include, but not limited to, alpha-phosphorothioate nucleotide triphosphates, morpholino triphosphates, peptide nucleic acids, peptide nucleic acid analogs, and sugar modified nucleotide triphosphates and combinations thereof.

The term “pre-defined” refers to defined, or established in advance.

The terms “protect” and “protected” refers to keeping safe or shielded from unwanted treatment by an enzyme, including but not limited to, digestion and/or another chemical, physical or mechanical means of treatment or exposure.

The terms “specific” and “specificity” refers to quality of belonging or relating uniquely to a target DNA sequence or DNA sequence fragment.

The term “sequence specificity” refers to a clearly defined region of a DNA or RNA chain/sequence.

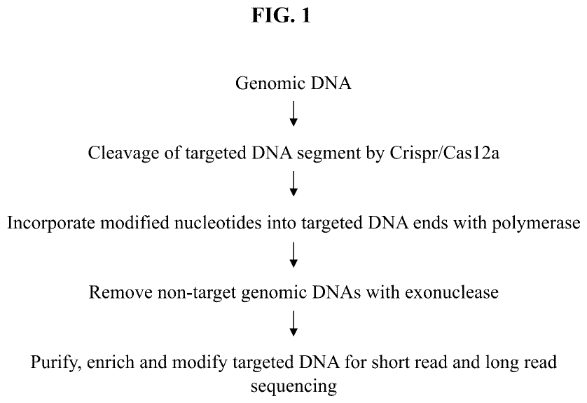

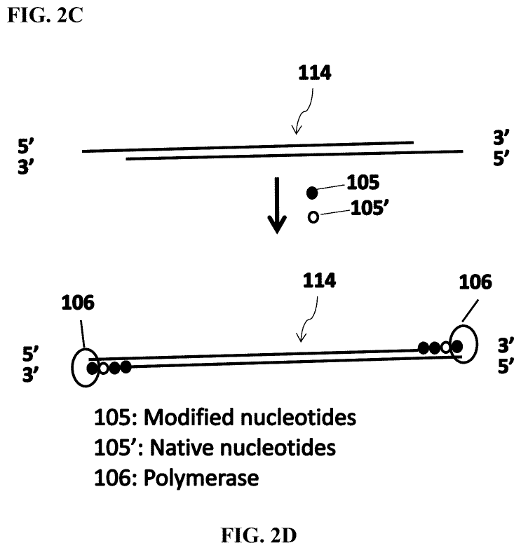

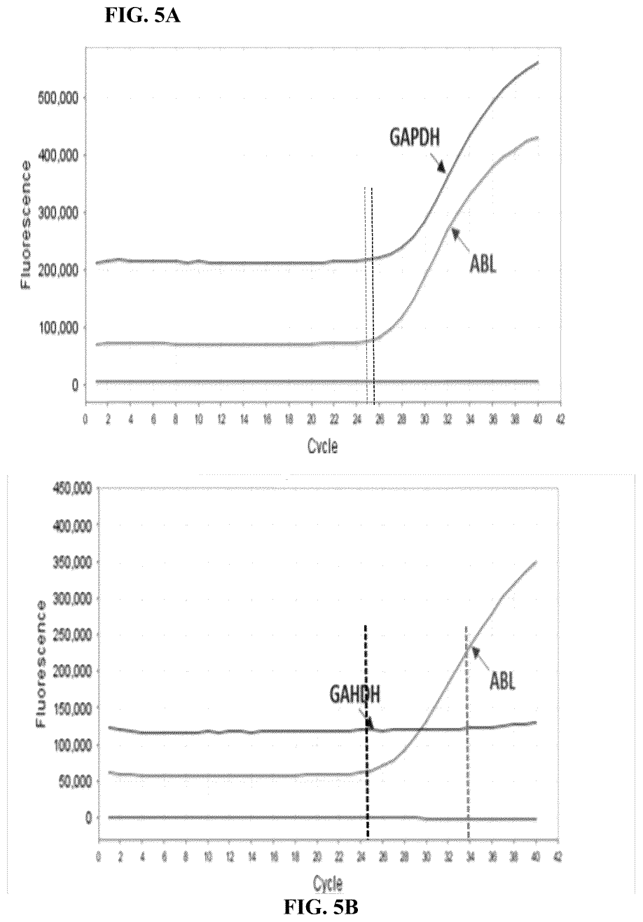

In one embodiment disclosed is a method of target DNA enrichment. In a single sample having at least one target DNA fragment sequence and at least one specific DNA nucleases with targeting oligonucleotides (ON) that are homologous to their respective selected binding sites on a target double-stranded DNA (“dsDNA”) (as illustrated in FIGS. 2A-2D). As used herein, homologous indicates that the targeting ON are complementary to one strand on the target dsDNA, and is thus capable of forming a triple helix with that target dsDNA, or forming a double helix with the complementary single DNA strand. The sequence specific DNA nuclease binds toa target DNA at the binding site, forming a target DNA-DNA nuclease complex and the sequence specific DNA nuclease cuts the target DNA at a cleavage point that is on or near the targeting oligonucleotide's binding site of the target dsDNA. After the target DNA was cut at both 5′ and 3′ ends (of complementary strands when dsDNA or only the 5′ or 3′ strand if single-stranded DNA), a polymerase was used to incorporate native (FIG. 2D, 105′) and modified nucleotide bases (FIG. 2D, 105), including, but not limited to for example, alpha-phosphorothioate nucleotide triphosphates, morpholino triphosphates, peptide nucleic acids, peptide nucleic acid analogs, and sugar modified nucleotide triphosphates. The modified nucleotide base(s) were attached to both ends of the fragments of interest by a polymerase (FIG. 2D, 106), the target fragment being dsDNA and to a single-strand if ssDNA. These modified bases were capable of protecting the DNA from DNA exonuclease digestion. The non-target linear DNAs with the sample were not protected at both ends by the modified nucleotide bases and thus, unprotected, non-target DNAs were digested by exonucleases. This resulted in enrichment of the target DNA fragments of interest in the final reaction mix (as detailed in FIG. 1 and illustrated in FIGS. 3 and 5).

In one aspect of the embodiment, as shown in FIG. 2, the reaction includes a targeting oligonucleotide 104 and 104′that is a guide RNA or analogs thereof. The nuclease 102 and 102′ includes CRISPR associated protein 12a (Cas12a) or variant(s) thereof (Advancedseq LLC, Livermore, CA U.S.A.). The targeting oligonucleotides direct the Cas12a protein or variant thereof to introduce double-stranded breaks in target DNA 110. The use of Cas12a protein 102 or 102′ as a programmable sequence specific DNA endonuclease is described in Ledford H. “Bacteria yield new gene cutter”. Nature. 526 (7571): 17. doi:10.1038/nature.2015.18432. PMID 26432219, (October 2015), which is incorporated herein by reference.

As used herein, a Cas12a variant includes a mutant of Cas12a that maintains part or all of Cas12a functions, or a Cas 12a homolog derived from a common ancestor that performs the same or similar function as Cas12a. As illustrated in FIG. 2B the target DNA 112 is cleaved at both ends by incubating with the CRISPR/Cas12a or variants thereof. The targeting oligonucleotide resides in a suitable buffer (10-100 mM Tris, pH 6-8) at a suitable temperature (25-42C° for a suitable length of time (5 minutes-4 hours). In a preferred embodiment, target DNA 112 is released from other non-target DNAs 108, 108′ (as illustrated in the FIG. 2B).

In yet another aspect of the embodiment, multiple pairs of sequence specific DNA nucleases are employed in one reaction. Thus, multiple, different sequence specific DNA fragments covering multiple, sequence specific regions of DNA sequences of interest may be cut and isolated in a multiplex reaction within a single vial.

In another aspect of the embodiment, multiple pairs of sequence specific DNA nucleases are employed in one reaction to cut out the same DNA sequence of interest from the same target DNA, but at different cutting points, resulting in multiple fragments of interest all including the same DNA region of interest. By carrying out such redundant cuts for the same DNA sequence of interest, the overall efficiency, i.e. percentage of target DNA cut, may be increased. By combining the forgoing two embodiments as illustrated in FIGS. 1 and 2, multiple DNA fragments covering the same DNA sequence of interest, as well as multiple DNA sequences of interest, may be cut and isolated in one run.

As Cas12a endonuclease generates cohesive ends after cleavage, modified nucleotides are added to the cohesive ends of the cleaved DNA segment of interest by polymerases 106 (FIG. 2D) for enrichment as the step next. A number of polymerases are capable of incorporating modified nucleotide bases complementary to the template strands of the original DNA. Those include, but are not limited to, Phusion DNA polymerase, Taq DNA polymerase, Vent DNA polymerase, Bst DNA polymerase, phi29 DNA polymerase, Sulfolobus DNA polymerase IV, Therminator DNA polymerase, DNA polymerase I, Klenow Fragment, T4 DNA polymerase, T7 DNA polymerase, Bsu DNA polymerase, and terminal transferase.

The modified nucleotides can include, but are not limited to, triphosphates comprising alpha-phosphorothioate nucleotide triphosphates, morpholino triphosphates, peptide nucleic acids, peptide nucleic acid analogs, and/or sugar modified nucleotide triphosphates.

In the enrichment step, non-modified DNAs are digested with exonucleases, including but not limited to, exonuclease III, T7 exonuclease, exonuclease V, exonuclease VIII, Lambda exonuclease, T5 exonuclease, nuclease Bal-31, as well as variants and truncated forms.

All publications, published patent documents, and patent applications cited herein are hereby incorporated by reference to the same extent as though each individual publication, published patent document, or patent application was specifically and individually indicated as being incorporated by reference.

### EXAMPLES

### Example I

Enrichment of Plasmid pGEM3Zf DNA With the Claimed Method

This scheme has been first experimentally demonstrated in the plasmid pGEM-3Zf. In an experiment as illustrated in FIG. 3, pGEM-3Zf plasmid was digested with CRISPR/Cas12a in the presence of two guide RNAs to release two DNA fragments of 0.7 and 2.5 kb, respectively (lane 1). The guide RNAs are complimentary to the target genomic DNA fragment's ends that are more than eight base pairs long so the probability of finding a matching complimentary genomic DNA sequence is one in 262144 (4⁹=262144) bases. The 5′-overhang ends of the cleaved fragments were filled in by incorporating dNTPs with Taq DNA polymerase for 30 min. at 72° C. (data not shown) to the cohesive ends. The modified fragments were then subjected to exonuclease III digestion. To confirm that exonuclease was able to digest away unprotected DNA, purified phage λ DNA (0.5 μg) was added to the reaction mixture before exonuclease treatment (lane 2).

FIG. 3, lane 3 illustrates that the protected ends are not digested by exonuclease digestion. The modified fragments plus phage λ DNA were then subjected to exonuclease digestion (lane 3). This resulted in complete digestion of phage λ DNA when exonuclease was added but pGEM-3Zf plasmid 0.7 kb and 2.5 kb fragments remained undigested. This result confirmed that the disclosed and claimed sequence-specific DNA enrichment method works effectively with plasmid DNA.

### Example II

Isolation of ABL1 as the Target Gene From Human Genomic dsDNA Using With the Claimed Method

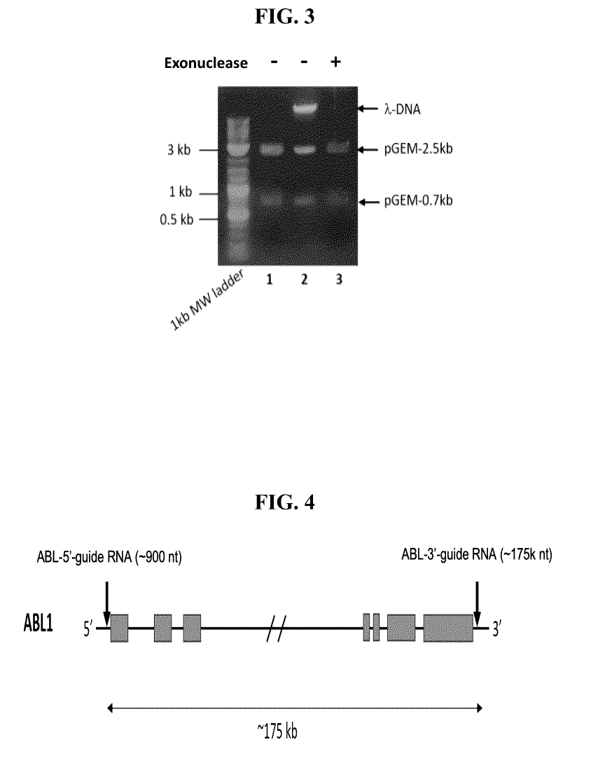

To demonstrate that the CRISPR/Cas12a based enrichment strategy can be used to selectively enrich long DNA sequences from the actual human genome, we selected human ABL1 as the target gene for the confirmatory experiments. ABL1 gene is approximately 175-kb long, consisting of multiple introns and exons segments as illustrated in FIG. 4. As a proto-oncogene that encodes a protein tyrosine kinase the ABL1 gene is involved in various cellular processes including cell division, adhesion, differentiation, and response to stress. Significantly, ABL1 has also been found fused to several translocation partner genes, most notably the breakpoint cluster region gene (BCR). BCR-ABL fusion proteins have been found in many forms of leukemia including in most cases of chronic myeloid leukemia (CML). As a result, ABL1 gene has become a desirable disease biomarker and therapeutic target for CML and other related leukemia.

To cleave ABL1 gene from within human genomic DNA, 1 μg total DNA was isolated from cultured HEK293 cells treated with CRISPR/Cas12a and ABL-5′-guide RNA and ABL-3′-guide RNA complexes for 30 min at 37° C. After the genomic DNA was cleaved with CRISPR-based Cas12a/guide RNA complexes, a mixture of dNTP analogues at final concentrations of 25 μM each and 10 Units of Taq DNA polymerase were added to the cleavage reaction mixture. After 30 min. incubation at 72° C., the reaction products were purified with ZYMO DNA Clean Kit (ZYMO Research, Tustin, CA, USA) by following the manufacturer's instructions. Purified DNA products were eluted with TE buffer to a final concentration of 20 ng/μl.

After the 5′-overhang ends of the CRISPR/Cas12a cleaved DNA fragments were filled in with alpha-phosphorothioate deoxyribonucleotides and purified with ZYMO DNA Clean Kit, the isolated DNA products were subjected to treatment with exonuclease III (New England Biolabs) following the manufacturer's instructions. Each reaction was carried out in a total volume of 20 μl containing ˜0.1 μg of isolated DNA and 50 Units of exonuclease III. The reaction mixtures were incubated for 30-60 min. at 37° C. followed by incubation at 70° C. for 15 min. to inactivate remaining exonuclease activity. The final products were then analyzed by real-time quantitative PCR assays for genomic DNA samples.

In the TaqMan real-time qPCR assay, each qPCR reaction consists of 5 min. initial incubation at 94° C. followed by 40 amplification cycles of 10 seconds at 94° C., and 40 seconds at 60° C. per cycle. The qPCR has sequence-specific primers/probe for ABL1 (probe labeled with Cy5) and housekeeping gene GAPDH (probe labeled with FAM) probes, respectively, so the assay is able to detect both genes simultaneously within a single reaction. As shown in FIG. 5A, signals for both ABL1 (purple-middle trace) and GAPDH gene (blue-upper trace) were detected simultaneously in the same TaqMan qPCR reaction before exonuclease III treatment. The Ct value for ABL1 is 25 and Ct for GAPDH is 25.6. As expected, after the DNA was treated with exonuclease III, only the signal for ABL1 gene was un-affected in the multiplexing assay (Ct=25) whereas the un-protected GAPDH gene lost most of its signal (Ct=34) (FIG. 5B). Ct values were determined from the point of increase in the trace for each of ABL1 and GAPDH as noted by the vertical lines crossing the traces in both FIGS. 5A and 5B.

The relative changes in Ct value between the TaqMan assays (ΔCt=0.4 for ABL1 and ΔCt=9 for GAPDH with and without exonuclease treatment) were determined by Ct values from 5A and 5B, As the Ct value of GAPDH difference is greater than 8, it was estimated that there is at least 100-fold enrichment for ABL1 relative to GAPDH in this experiment (2⁸=256 fold difference as each unit of Ct value difference represents 2 fold difference in concentration). These results demonstrated the claimed method can isolate and enrich large genomic dsDNA segments from human genomic DNA efficiently.

In a further embodiment, the target DNAs can be modified in the enrichment step by adding linkers or tags including, but not limited to, e.g., biotin or another affinity tag used to bind the target DNA to a solid support and pull down the target DNA from the reaction solution mix. The resulting isolated target DNA can undergo further purifications and manipulations for e.g. sequencing analysis, as is known to the skilled artisan.

While embodiments and applications of this disclosure have been shown and described, the terms, and the following claims should not be construed to limit the claims to the disclosed specification's specific embodiments. It would be apparent to those skilled in the art that many more modifications and improvements than mentioned above are possible without departing from the inventive concepts herein. All possible embodiments along with the full scope of equivalents should be construed for the disclosed terms and to which such claims are entitled such that the claims are not limited by the disclosure.

What is claimed is:

1. A method for improving specificity for enrichment of a target DNA comprising:
a. providing a genomic target DNA sample comprising one or more double-stranded (DS) target fragments within at least one target region, wherein the at least one target region comprises one or more double-stranded target DNA fragments;
b. cleaving both ends of the one or more double-stranded target fragment(s), with a CRISPR/Cas12a-guide RNA (gRNA) endonuclease complex, wherein the endonuclease is a sequence specific DNA nuclease; and wherein the gRNA is complementary to each flanking region of its respective end within or outside the one or more target fragment(s) to be enriched to create cohesive 5′ over-hanging single-stranded ends;
c. incorporating modified nucleotides (ON) using a DNA polymerase to each of the 5′ overhanging ends of the one or more DS target fragment(s), wherein the modified nucleotides protect the one or more DS target fragment(s) from exonuclease digestion; and
d. digesting the unprotected genomic DNA with an exonuclease, wherein the one or more target DS fragment(s) are specifically enriched.

2. The method of claim 1, wherein each of the Cas12a-gRNA complexes comprises a Cas12a protein and a Cas12a-associated gRNA that are complimentary to different, pre-defined, sites of the targeted genomic DNA.

3. The method of claim 1, wherein the sequence specific DNA nuclease is capable of cutting a target double stranded DNA (dsDNA) with sequence specificity as defined by the guide RNAs that are complimentary to the target genomic DNA fragment's ends that are more than eight base pairs long so the probability of finding a matching complimentary genomic DNA sequence is one in 262144 (4⁹=262144) bases.

4. The method of claim 1, wherein said sequence specific DNA nuclease with its associated guide RNAs is capable of generating cohesive ends of cut double-stranded target DNA fragment(s).

5. The method of claim 1, wherein said DNA polymerase is capable of modifying cohesive ends of cleaved target DNA fragments by ligating the modified ONs to the cohesive ends, thereby protecting the target DS fragment(s) from DNA exonuclease digestion.

6. The method of claim 1, wherein said exonuclease is capable of enriching target DNA fragment(s), wherein the exonuclease(s) digests non-target DNA regions and fragments not protected at both ends by modified ONs.

7. The method of claim 1, wherein enriched target DNA fragment(s) can be further modified for purification for sequence analysis.

8. The method of claim 1, wherein said modified nucleotides are selected from the group consisting of alpha-phosphorothioate nucleotide triphosphates, morpholino triphosphates, peptide nucleic acids, peptide nucleic acid analogs, and sugar modified nucleotide triphosphates and combinations thereof.

9. The method of claim 7, wherein the enriched target DNA fragment(s) are further purified for sequence analysis.

10. The method of claim 1, wherein said sequence specific DNA nuclease comprises a Cas12a protein or a variant thereof.

11. The method of claim 1, wherein guide RNAs are single-stranded DNAs or RNAs.

12. The method of claim 1, wherein each of the gRNAs are between 15-100 nucleotides in length and wherein the 10-50 nucleotide long sequence is complementary to a sequence on one strand of each end of the target DNA fragment.

13. The method of claim 1, further comprising at least one pair of targeting oligonucleotides, and the composition causes the target DNA to be cut by said sequence specific DNA nuclease at both ends of the DNA fragment of interest in such a way that both ends are cohesive.

14. The method of claim 4, where the cohesive end generated by sequence specific DNA nuclease cut has 5′ overhangs.

15. The method of claim 1, further comprising at least one polymerase and modified nucleotides, and the polymerase is capable of incorporating modified nucleotides at the cohesive ends of the target DNAs.

16. The method of claim 1, further comprising at least one ligase and double stranded DNA or RNA linkers with modified nucleotides or hairpin linkers, and the ligase and linkers are capable of being ligated to the ends of the enriched target DNAs.

17. A kit comprising:
a. a sequence specific DNA nuclease;
b. a DNA polymerase;
c. modified nucleotides;
d. DNA exonuclease; and optionally
e. a ligase.

18. A DNA enrichment kit comprising at least two of the following reagents:
a. a Cas12a protein or a variant thereof;
b. a targeting oligonucleotide (guide-RNA);
c. a DNA polymerase;
d. modified nucleotides;
e. a DNA exonuclease;
f. a DNA or RNA ligase; and
g. DNA/RNA linkers.

19. The reagent kit of any one of claims 17 and 18, wherein said modified nucleotides is selected from the group consisting of alpha-phosphorothioate nucleotide triphosphates, morpholino triphosphates, peptide nucleic acids, peptide nucleic acid analogs, or sugar modified nucleotide triphosphates and combinations thereof.

20. The reagent kit of any one of claims 17 and 18, wherein said exonuclease is selected from the group of exonuclease III, T7 exonuclease, exonuclease V, exonuclease VIII, Lambda exonuclease, T5 exonuclease, nuclease Bal-31, their variants and truncated forms.
